(19) United States
(12) Patent Application Publication
(10) Pub. No.: US 2024/0286997 A1
(43) Pub. Date: Aug. 29, 2024

(54) METHOD FOR PREPARING MERCAPTANS BY SULFHYDROLYSIS OF DIALKYL SULFIDES WITH CATALYST PRETREATMENT

(71) Applicant: ARKEMA FRANCE, Colombes (FR)

(72) Inventors: Hélori SALEMBIER, Lacq (FR); Georges FREMY, Lacq (FR); Guillaume LAUDUMIEY, Lacq (FR); Louis CORBEL DEMAILLY, Lacq (FR)

(73) Assignee: ARKEMA FRANCE, Colombes (FR)

(21) Appl. No.: 18/566,958

(22) PCT Filed: Jun. 20, 2022
(86) PCT No.: PCT/FR2022/051192

§ 371 Date: Dec. 4, 2023

(30) Foreign Application Priority Data
Jun. 21, 2021 (FR) FR2106567

(51) Int. Cl. C07C 319/06

(52) U.S. Cl. CPC C07C 319/06

(57) ABSTRACT

The present invention relates to a process for preparing at least one mercaptan, comprising the following steps:
i) treatment of a catalyst for the sulfhydrolysis of at least one dialkyl sulfide, preferably a zeolite, said treatment comprising the following steps:
   1) heating said catalyst, preferably in the presence of an inert gas; and
   2) placing said catalyst in contact with hydrogen sulfide (H₂S); and then
ii) a sulfhydrolysis reaction in which at least one dialkyl sulfide is reacted with H₂S in the presence of said catalyst treated according to step i), to obtain at least one mercaptan;
said catalyst being a zeolite.

The present invention relates to a process for preparing mercaptans, in particular methyl mercaptan, from dialkyl sulfide(s) and hydrogen sulfide (also known as the sulfhydrolysis process or reaction), in the presence of a catalyst which has undergone a pretreatment. The present invention also relates to a process for preparing mercaptans and dialkyl sulfides, from at least one alcohol and hydrogen sulfide, involving the sulfhydrolysis process as defined above.

Mercaptans are of great interest industrially and are currently in widespread use in the chemical industries, notably as starting materials in the synthesis of more complex organic molecules. For example, methyl mercaptan (CH₃SH) is used as a starting material in the synthesis of methionine, an essential amino acid for animal nutrition. Methyl mercaptan is also used in the synthesis of dialkyl disulfides, in particular in the synthesis of dimethyl disulfide (DMDS), a sulfiding additive for hydrotreating catalysts for petroleum fractions, among other applications.

Mercaptans, and in particular methyl mercaptan, are generally synthesized industrially by a known process starting from an alcohol and hydrogen sulfide at elevated temperature in the presence of a catalyst according to equation (1) below:

### Main Reaction

$$R OH + {H}_{2} S -> R SH + {H}_{2} O \qquad ( 1 )$$

However, this reaction gives rise to the formation of byproducts, such as dialkyl sulfides (which are symmetrical in the case below) according to equation (2) below:

$$R OH + R SH -> R S R + {H}_{2} O \qquad ( 2 )$$

Furthermore, when the main reaction is performed in the presence of several alcohols, dissymmetrical dialkyl sulfides may also be obtained according to equations (3) and (4) below (example given with two alcohols):

$$R OH + {R}^{′} OH + 2 {H}_{2} S - > R SH + {R}^{′} SH + 2 {H}_{2} O \qquad ( 3 )$$

$$R OH + {R}^{′} SH -> {R S R}^{′} + {H}_{2} O \qquad ( 4 )$$

The symmetrical or dissymmetrical dialkyl sulfide byproducts are obtained in large amount industrially and are primarily sent for destruction. This represents a loss of efficiency in the mercaptan production process and an added cost associated with destroying them.

Dialkyl sulfides are occasionally upgraded to obtain the corresponding mercaptans, by means of the following reaction (5) (also known as sulfhydrolysis):

### Sulfhydrolysis Reaction

$${R S R}^{′} + {H}_{2} S -> R SH + {R}^{′} SH \qquad ( 5 )$$

In the case of methyl mercaptan, the sulfhydrolysis reaction is written according to equation (6) below:

$$C {H}_{3} S C {H}_{3} + {H}_{2} S - > 2 C {H}_{3} SH \qquad ( 6 )$$

The sulfhydrolysis reaction is generally catalyzed with catalysts of alumina type (Al₂O₃) or of NiMo (nickel/molybdenum) or CoMo (cobalt/molybdenum) type on an alumina support, as described in patent applications WO 2017/210070 and WO 2018/035316.

U.S. Pat. Nos. 4,396,778 and 4,313,006 describe the use of zeolites as catalysts for the sulfhydrolysis reaction. However, the zeolites described are used directly and are not pretreated. Furthermore, the operating conditions of the tests performed are not suitable for industrial production (notably with low productivities).

Such a sulfhydrolysis process thus needs to be improved so as to be more efficient and more suited to the industrial scale.

There is thus a need for an improved process for the sulfhydrolysis of dialkyl sulfides to mercaptans, in particular of dimethyl sulfide to methyl mercaptan.

There is also a need for an improved process for upgrading dialkyl sulfides, in particular dimethyl sulfide, which are generated as byproducts during the production of mercaptans produced from alcohol(s) and hydrogen sulfide.

One objective of the present invention is to propose a process for the sulfhydrolysis of dialkyl sulfides to mercaptans which is efficient and easy to perform industrially, notably with an improved catalyst.

Another objective of the present invention is to propose a process for the sulfhydrolysis of dialkyl sulfides to mercaptans which can be readily integrated into a unit for the industrial production of mercaptans, notably produced from alcohol(s) and H₂S.

One objective of the present invention is to provide an integrated process for preparing mercaptans in which the dialkyl sulfides generated as byproducts (for example during the reaction between an alcohol and H₂S) are recycled or economically upgraded in a manner that is industrially viable, easy and safe for the operators.

The present inventors have discovered, surprisingly, that pretreatment of the catalysts used for sulfhydrolysis allows improved conversion of dialkyl sulfides while at the same time maintaining high selectivity of the reaction for mercaptans. The yield and productivity of the process are thus improved. In particular, the dialkyl sulfide conversion is improved relative to the conversion obtained in the presence of the same untreated catalyst. An increase in conversion of at least 8%, or even at least 10%, is notably obtained by means of the pretreatment of the catalyst according to the invention.

The sulfhydrolysis process thus improved may be integrated into a plant for the industrial production of mercaptans, notably produced from at least one alcohol and H₂S (main reaction).

The sulfhydrolysis process according to the invention then makes it possible to increase the mercaptan productivity in a simple and economical manner by upgrading the dialkyl sulfides generated as byproducts during the main reaction and transforming them also into mercaptans.

Furthermore, the mercaptans derived from the sulfhydrolysis and the unreacted H₂S can be reintroduced directly (notably without an intermediate purification step) into the main reactor, and without this having any consequence on the reaction between the alcohol(s) and the H₂S.

The mercaptans produced by the two reactions (main reaction and sulfhydrolysis reaction) can then be purified and/or recovered in a single place, for example at the outlet of the main reactor.

This integration of the sulfhydrolysis process into the main mercaptan production chain can be reinforced by the presence of a single H₂S feed for both the main reaction and the sulfhydrolysis reaction (for example at the inlet of the sulfhydrolysis reactor).

Thus, according to the invention, a simple and efficient process for the upgrading of dialkyl sulfides which is totally integrated into an industrial mercaptan production chain may be obtained. This device is notably easy to implement: it can be readily connected to the main unit and requires only minimal modifications thereof.

Thus, the present invention relates to a process for preparing at least one mercaptan, comprising the following steps:
i) treatment of a catalyst for the sulfhydrolysis of at least one dialkyl sulfide, preferably a zeolite, said treatment comprising the following steps:
   1) heating said catalyst, preferably in the presence of an inert gas; and
   2) placing said catalyst in contact with hydrogen sulfide (H₂S); and then
ii) a sulfhydrolysis reaction in which at least one dialkyl sulfide is reacted with H₂S in the presence of said catalyst treated according to step i), to obtain at least one mercaptan.

The present invention also relates to a process for preparing at least one mercaptan, preferably continuously, comprising the following steps:
A) H₂S and at least one alcohol are introduced into a first reactor;
B) the H₂S and said at least one alcohol are reacted to obtain an outlet stream comprising at least one mercaptan and at least one dialkyl sulfide and possibly H₂S;
C) the outlet stream obtained from step B) is separated into:
   a stream F1 comprising the mercaptan(s),
   a stream F2 comprising the dialkyl sulfide(s), and
   optionally a stream F3 comprising H₂S;
D) optionally, the stream F2 is purified to obtain a stream F2′ enriched in dialkyl sulfide(s); E) stream F2 or F2′ and H₂S are introduced into a second reactor, said reactor comprising a catalyst treated according to the treatment process as defined above;
F) a sulfhydrolysis reaction of the dialkyl sulfide(s) with H₂S is performed to obtain an outlet stream F4 comprising said mercaptan(s) and possibly H₂S;
G) optionally, stream F4 obtained from step F) is recycled into step A).

### Definitions

The term “catalyst” notably means a substance or a composition of chemical substances which accelerate a chemical reaction and which are found unchanged at the end of this reaction.

The usual definitions of conversion and selectivity are as follows:

Conversion %=(number of moles of reagent in the initial state-number of moles of reagent in the final state)/(number of moles of reagent in the initial state)×100

Mercaptan selectivity=Number of moles of reagent converted into the desired product/X*(number of moles of reagent in the initial state-number of moles of reagent in the final state)×100

with X=2 if the dialkyl sulfide is symmetrical or X=1 if the dialkyl sulfide is dissymmetrical. The term “GHSV” (Gas Hourly Space Velocity), expressed in h⁻¹, is notably understood as follows:

$$GHSV = \frac{Q \cdot T \cdot {P}^{0}}{{V}_{c a t} \cdot P \cdot 273 , 15} \qquad [ Math 1 ]$$

with:
Q=inlet gas flow rate in NL/h (normo liters per hour)
T=reaction temperature in Kelvins
P⁰=standard pressure in bar
P=reaction pressure in bar
V_{cat}=catalyst volume (L).

In particular, pretreatment of the catalysts according to the invention make it possible to obtain a dialkyl sulfide conversion of between 30% and 90%, preferably between 40% and 80% and even more preferentially between 40% and 75%.

The selectivity of the sulfhydrolysis reaction for mercaptans is notably greater than or equal to 99%, or even greater than 99.5%.

Hereinbelow, it is understood that when two reagents are introduced into a reactor, they may each be introduced separately into the reactor or may be combined before being introduced into the reactor.

The expression “between X and X” includes the limits mentioned, unless mentioned otherwise.

### Sulfides, Disulfides and Mercaptans

The term “sulfide” notably means any organic compound comprising a —C—S—C— function.

The term “disulfide” notably means any organic compound comprising a —C—S—S—C— function.

The term “dialkyl sulfide” notably means a compound of general formula (I) below:

R—S—R′ (I)

in which R and R′, which may be identical or different, are, independently of each other, a saturated, linear, branched or cyclic, optionally substituted hydrocarbon-based radical.

Preferably, R and R′, which may be identical or different, are, independently of each other, a linear, branched or cyclic alkyl radical containing between 1 and 18 carbon atoms, preferably between 1 and 12 carbon atoms.

R and R′, which may be identical or different, may be chosen, independently of each other, from the group consisting of methyl, ethyl, propyl, butyl, pentyl, hexyl, heptyl, octyl, nonyl, decyl, undecyl and dodecyl (and also positional isomers thereof). Preferably, R and R′, which may be identical or different, may be chosen, independently of each other, from the group consisting of methyl, ethyl, octyl and dodecyl.

Preferably, R and R′ are identical (which corresponds to a symmetrical dialkyl sulfide). Symmetrical dialkyl sulfides in particular have the general formula (II) below:

R—S—R (II)

in which R is as defined above.

In particular, the dialkyl sulfides according to the invention are chosen from the group consisting of dimethyl sulfide, diethyl sulfide, dioctyl sulfide, didodecyl sulfide and methyl ethyl sulfide. The dialkyl sulfides according to the invention may be chosen from the group consisting of dimethyl sulfide, di-n-propyl sulfide, di-isopropyl sulfide, di-n-butyl sulfide, di-sec-butyl sulfide and di-isobutyl sulfide. Most particularly preferably, the dialkyl sulfide is dimethyl sulfide (DMS).

The mercaptans according to the invention are notably those corresponding to the sulfhydrolysis of the dialkyl sulfides as defined above. The term “mercaptans” notably means alkyl mercaptans.

In particular, the term “alkyl mercaptan” means a compound of general formula (III) or (IV) below:

R—SH (III) or R′SH (IV),

in which R and R′ are as defined for the general formula (I) above.

Particularly preferably, the mercaptan is methyl mercaptan.

In particular, the term “dialkyl disulfide” (also referred to below as DADS) means a compound of the general formula (V) below:

R—S—S—R′ (V)

in which R and R′ are as defined for the general formula (I) above.

In particular, the dialkyl disulfides according to the invention are chosen from the group consisting of dimethyl disulfide, diethyl disulfide, dioctyl disulfide, didodecyl disulfide and methyl ethyl disulfide. The dialkyl disulfides according to the invention may be chosen from the group consisting of dimethyl disulfide, diethyl disulfide, dioctyl disulfide and didodecyl disulfide. Most particularly preferably, the dialkyl disulfide is dimethyl disulfide (DMDS).

### Process for Preparing at Least One Mercaptan by Sulfhydrolysis

The present invention relates to a process for preparing at least one mercaptan, comprising steps i) and ii) as defined below.

Step i)—Treatment of the Catalyst for the Sulfhydrolysis of Dialkyl Sulfides to Mercaptans

The treatment (or pretreatment) of the catalyst for the sulfhydrolysis of at least one dialkyl sulfide, preferably a zeolite, comprises the following steps:
1) heating said catalyst, preferably in the presence of an inert gas; and
2) placing said catalyst in contact with hydrogen sulfide (H₂S).

Although it may be performed ex-situ (i.e. outside the reactor in which step ii) takes place), this treatment is preferably performed in-situ (i.e. in the reactor in which step ii) takes place).

The heating step 1) may be performed in the presence of an inert gas and at a temperature of between 70° C. and 350° C., more particularly between 80° C. and 250° C., and preferably between 80° C. and 150° C. Without wishing to be bound by theory, said heating step may correspond to a step of drying or dehydration of said catalyst.

The heating step 1) is notably performed in the presence of an inert gas. Preferably, the term “inert gas” means any gas which is inert (i.e. not chemically reactive) relative to said catalyst. Among the inert gases, mention may be made of dinitrogen (N₂), dry air, methane (CH₄), carbon dioxide (CO₂), natural gas, or gases from group 18 of the Periodic Table of the Elements (i.e. gases chosen from helium, neon, argon, krypton, xenon and radon). In particular, the heating is performed under nitrogen. In particular, the inert gas is not dihydrogen (H₂).

The heating step 1) may be performed at a temperature of between 70° C. and 350° C., more particularly between 80° C. and 250° C., and preferably between 80° C. and 150° C. A temperature ramp may be performed with a rise to about 70° C. and then a stepwise rise, for example in steps of 0.5° C. to 15° C., preferably between 5° C. and 10° C., per minute to the desired temperature.

The heating step 1) may be performed at a pressure of between 0.1 and 50 bar absolute, in particular between 0.1 and 10 bar absolute, preferably between 0.8 and 2 bar absolute.

The heating step 1) may last between 0.1 h and 24 h, in particular between 0.1 h and 5 h, for example about 1 h.

Monitoring of the heating step 1) may notably be performed by monitoring the temperature.

Step 2) of placing in contact with H₂S may be performed at a temperature of between 20° C. and 450° C., for example between 250° C. and 400° C., preferably between 320° C. and 370° C.

A temperature ramp may be performed, rising from 2° C. to 10° C. per minute until the desired temperature is reached.

A temperature ramp may also be performed with a rise from 5° C. to 50° C. per hour up to the desired temperature.

The HSV may be between 1 and 2000 h⁻¹, preferably between 1 and 1000 h⁻¹, more preferably between 1 and 700 h⁻¹.

The GHSV may be between 1 and 5000 h⁻¹, preferably between 1 and 2000 h⁻¹, more preferentially between 10 and 1500 h⁻¹.

Step 2) may be performed at a pressure of between 0.1 and 50 bar absolute, more particularly between 1 and 20 bar absolute, for example between 1 and 15 bar absolute or between 5 and 10 bar absolute.

Step 2) may last between 0.1 h and 48 h, preferably between 0.1 h and 15 h, more preferentially between 0.5 h and 15 h, for example about 1 h.

The placing in contact with H₂S may be performed with pure H₂S or as a mixture with an inert gas as defined above. When H₂S is mixed with an inert gas, the amount of H₂S may be between 0.1% and 100%, more particularly between 60% and 100%, preferably between 95% and 100% by volume relative to the volume of inert gas. The inert gas is preferentially the same as that used in step 1).

This step 2) may be performed with an H₂S concentration gradient. H₂S, either pure or as a mixture, may notably be introduced continuously, preferably into the reactor in which the sulfhydrolysis is performed. The H₂S flow rate may be between 100 and 2000 kg/h.

Preferably, steps 1) and 2) are performed successively. The treatment according to the invention may consist of steps 1) and then 2). The catalyst treatment as described above is notably performed when changing and/or regenerating said catalyst.

In particular, said treatment does not correspond to a conventional pre-sulfurizing or sulfurizing treatment of a catalyst used for hydrotreating (known as “sulfurizing” or “sulfiding”). To sulfurize hydrotreating catalysts, it is known practice to place said catalysts in contact with H₂S and H₂ (a sulforeduction is performed). Thus, the treatment according to the invention preferably does not comprise the introduction or addition of dihydrogen (H₂), either into step 1) and/or 2). More particularly, step 2) does not comprise dihydrogen as a reagent. More specifically, step 2) is performed substantially in the absence of dihydrogen. For example, dihydrogen may be present in a content of less than 100 ppmv.

Such a process notably enables the catalyst performance to be improved by increasing the conversion of the dialkyl sulfide(s). Such a treatment may be called a pretreatment, preactivation or activation. The treated catalyst may be previously active or inactive: the treatment can enable its properties to be improved, such as improving the conversion and/or selectivity of the sulfhydrolysis reaction, or may allow it to be activated.

Any type of catalyst allowing the sulfhydrolysis reaction to be catalyzed may thus be treated. Mention may notably be made of catalysts, whether promoted or non-promoted, based on zeolites, alumina (Al₂O₃), silica (SiO₂), titanium dioxide (TiO₂), aluminosilicate, bentonite or zirconia (ZrO₂). These catalysts comprise or may consist of zeolites, alumina (Al₂O₃), silica (SiO₂), titanium dioxide (TiO₂), aluminosilicate, bentonite or zirconia (ZrO₂) and optionally one or more promoters.

The term “promoter” (also known as a “dopant”) notably means a chemical substance or a composition of chemical substances which can modify and notably improve the catalytic activity of a catalyst. For example, the term “promoter” means a chemical substance or a composition of chemical substances for improving the conversion and/or selectivity of the catalyzed reaction relative to the catalyst alone. Such substances are well known, for example alkali metals, nickel (Ni), molybdenum (Mo), cobalt (Co), tungsten (W) or combinations thereof (for example, NiMo and CoMo combinations). It is understood that these promoters may be in their oxide or sulfide form (for example, sodium may be in the oxidized Na₂O form). Preferably, the promoter is chosen from alkali metal oxides, in particular Na₂O.

The term “alkali metal” notably means lithium, sodium, potassium, rubidium and cesium, preferably sodium.

In particular, said catalyst comprises less than 10% by weight of promoter, more preferentially less than 2% by weight of promoter, relative to the total weight of the catalyst. Said catalyst may comprise between 0% and 10% by weight of promoter, preferably between 0% and 2% by weight of promoter, for example between 0.01% and 2% by weight of promoter, relative to the total weight of said catalyst.

The following catalysts may thus be treated:
promoted or non-promoted zeolites; preferably zeolites of type X, Y or L, more preferentially zeolites Y;
promoted or non-promoted alumina-based catalysts;
mention may be made in particular of catalysts based on alumina, based on NiMo (nickel/molybdenum) and/or CoMo (cobalt/molybdenum) supported on alumina, based on cadmium sulfide supported on alumina, based on tungsten trisulfide supported on alumina, based on alumina promoted with at least 1% by weight of alkali metal oxide, or else based on non-promoted alumina, for example gamma-alumina (such catalysts are notably described in patent applications WO 2018/035316, WO 2017/210070 and US 2008/0200730);
promoted or non-promoted silica (SiO₂) catalysts;
promoted or non-promoted titanium dioxide (TiO₂) catalysts, (notably as described in patent application FR3101631);
promoted or non-promoted aluminosilicate-based catalysts;
promoted or non-promoted bentonite-based catalysts, for example promoted with at least 1% by weight of an alkali metal oxide, as described in US 2008/0200730; and promoted or non-promoted zirconia-based catalysts (notably as described in patent application FR 3101631).

Preferably, the catalyst according to the invention is a promoted or non-promoted zeolite of the X, Y or L type, more preferentially of the Y type.

A zeolite is a crystal formed from an aluminosilicate micropore skeleton or support, whose connected void spaces are initially occupied by cations and water molecules.

The zeolites according to the invention in particular have a lattice parameter of between 24.30 and 24.70 Å and/or an Si/Al ratio of between 2.5 and 15.

Mention may thus be made of the zeolites sold by the company Axens under the name TCC101®.

In particular, said zeolite comprises less than 10% by weight of alkali metal oxide, more preferentially less than 2% by weight of alkali metal oxide, relative to the total weight of the zeolite. In particular, said alkali metal oxide is sodium oxide (Na₂O).

Most particularly preferably, said catalyst is a Y-type zeolite comprising between 0% and 10%, preferably between 0.01% and 10%, more preferentially between 0.01% and 2% by weight of an alkali metal oxide (preferably Na₂O), relative to the total weight of the zeolite.

The initial zeolite cation, for example sodium, may have been totally or partially replaced with at least one other cation using techniques known for zeolites, for example chosen from the group consisting of (with the exception of the cation to be replaced in the list below): H, Li, Na, K, Mg, Ca, Cs, Ba, La, Zr, Mo, W, Mn, Re, Fe, Ru, Co, Rh, Ni, Pd, Pt, Cu, Zn, Ag, Sn and Ga. These cation exchanges may be performed via conventional techniques.

For example, the zeolites may be treated in ammonium form. This involves calcination in the presence of steam. The zeolite in ammonium form (NH₄⁺) is then converted into protonic form (H⁺) by heating. Steam treatment hydrolyzes the Si—O—Al bonds. Aluminum migrates into the micropore volume in the form of aluminic debris. Simultaneously with the creation of these aluminic or silicoaluminic species, part of the lattice collapses, thus creating a mesoporosity. The silicon in these parts of the lattice is then transported to the vacant sites by the steam.

Such techniques are notably explained in the publication by Christine E. A. Kirschhock, Eddy J. P. Feijen, Pierre A. Jacobs, Johan A. Martens; first published: 15 Mar. 2008 https://doi.org/10.1002/9783527610044.hetcat0010 (Part 2. Preparation of Solid Catalysts; 2.3. Bulk Catalysts and Supports; 2.3.5 Hydrothermal Zeolite Synthesis).

The catalysts according to the invention may comprise stabilizers and/or binders. The stabilizers and binders are those conventionally used in the field of catalysts.

Following this treatment, the sulfhydrolysis reaction can be performed, in which at least one dialkyl sulfide is reacted with H₂S in the presence of the treated catalyst, notably allowing a better conversion of the dialkyl sulfide(s) to be obtained, relative to a reaction performed with said untreated catalyst.

Step ii)—Sulfhydrolysis Reaction of at Least One Dialkyl Sulfide to Mercaptan

Step ii) relates to the sulfhydrolysis reaction in which at least one dialkyl sulfide is reacted with H₂S in the presence of said catalyst treated according to step i), to obtain at least one mercaptan.

The sulfhydrolysis reagents may be in gaseous, liquid or solid form, preferably gaseous or liquid form, under the reaction temperature and pressure conditions.

The sulfhydrolysis reaction temperature may be between 100° C. and 500° C., preferably between 200° C. and 400° C., more preferably between 200° C. and 380° C. and more preferentially between 250° C. and 380° C.

The sulfhydrolysis reaction may be performed at a pressure of between 50 mbar and 100 bar absolute, preferably between atmospheric pressure (about 1 bar) and 50 bar absolute, and advantageously between 5 and 20 bar absolute.

The H₂S/dialkyl sulfide mole ratio may be between 0.1/1 and 50/1, preferably between 2/1 and 20/1. Preferably, said ratio is between 2/1 and 15/1, more preferably between 2/1 and 8/1, for example between 2/1 and 6/1, such as 4/1.

The flow rate of the dialkyl sulfide into the reactor in which the sulfhydrolysis takes place may be gradual.

Advantageously, the reagents (dialkyl sulfide(s) and H₂S) may respect a particular contact time with the catalyst. This parameter is expressed with the hourly space velocity equation:

(HSV)=(total CNTP gas flow rate by volume of dialkyl sulfide+H₂S entering)/(volume of catalyst in the reactor).

The HSV may be between 100 and 1200 h⁻¹.

The GHSV (Gas Hourly Space Velocity) may be between 1 and 100 000 h⁻¹, preferably between 100 and 10 000 h⁻¹, more preferentially between 100 and 3000 h⁻¹.

The sulfhydrolysis reaction may take place in any type of reactor, for example fixed-bed tube reactors, multitubular reactors, with microchannels, with a catalytic wall or with a fluidized bed, preferably a fixed-bed tube reactor.

The amount of each reagent supplied to the reactor may vary as a function of the reaction conditions (for example the temperature, the hourly space velocity, etc.) and is determined according to the general knowledge. The hydrogen sulfide may be present in excess.

Integrated Process for Preparing at Least One Mercaptan from at Least One Alcohol and H₂S

The present invention also relates to a process for preparing at least one mercaptan, comprising the steps of:
preparing mercaptan(s) and dialkyl sulfide(s) from at least one alcohol and H₂S, and then
sulfhydrolysis reaction of said dialkyl sulfide(s) according to step ii) as defined above in the presence of a catalyst treated according to step i) as defined above.

The reaction between an alcohol and H₂S to form a mercaptan and water is a known reaction, described, for example, in U.S. Pat. Nos. 2,820,062, 7,645,906 B2 and 2,820,831. For example, the reaction may be performed at a temperature of between 200° C. and 450° C. and/or at a pressure ranging from a reduced pressure to 100 bar. Generally, a catalyst is present, such as alumina promoted with alkali metals and/or alkaline-earth metals. The H₂S may be present in excess.

Among the reagents, at least one alcohol, preferably one or two alcohols, may be used. Preferentially, only one alcohol is used. The alcohol(s) may notably be chosen from (C₁-C₁₈) or even (C₁-C₁₂) alkane alcohols, and mixtures thereof. In particular, the alcohols may be chosen from the group consisting of methanol, ethanol, octanol, dodecanol and mixtures thereof. Preferably, the alcohol used is methanol.

On conclusion of this step, an outlet stream comprising at least one mercaptan, at least one dialkyl sulfide (as byproduct) and possibly H₂S is recovered. The outlet stream may also comprise water.

The present invention relates in particular to a process for preparing at least one mercaptan, preferably continuously, comprising the following steps:
A) H₂S and at least one alcohol are introduced into a first reactor;
B) the H₂S and said at least one alcohol are reacted to obtain an outlet stream comprising at least one mercaptan and at least one dialkyl sulfide and possibly unreacted H₂S;
C) said outlet stream obtained from step B) is separated into:
   a stream F1 comprising the mercaptan(s),
   a stream F2 comprising the dialkyl sulfide(s), and
   optionally a stream F3 comprising H₂S;
D) optionally, stream F2 is purified to obtain a stream F2′ enriched in dialkyl sulfide(s);
E) stream F2 or F2′ is introduced with H₂S into a second reactor, said reactor comprising a catalyst treated according to the treatment as defined above (i.e. the treatment according to step i) above);
F) a sulfhydrolysis reaction of the dialkyl sulfide(s) with H₂S is performed to obtain an outlet stream F4 comprising said mercaptan(s) and possibly unreacted H₂S;
G) optionally, stream F4 obtained from step F) is recycled into step A).

The term “stream F2′ enriched in dialkyl sulfide(s)” notably means a stream which comprises a weight percentage of dialkyl sulfide(s) (relative to the total weight of said stream F2′) greater than the weight percentage of dialkyl sulfide(s) relative to the total weight of said stream before said purification step (i.e. of the stream F2).

The outlet stream F4 from the second reactor of step F) may be recycled, partially or entirely, into the first reactor of step A). In particular, stream F4 may correspond entirely or partially, preferably entirely, to the stream comprising H₂S from step A), optionally with stream F3 comprising H₂S obtained from step C).

Preferably, streams F1 and F2 are liquid and/or stream F3 is gaseous. Stream F3 may be totally or partially:
recycled into step A) and/or recycled into step E) and/or
combined with stream F1 or F2 or F2′.

The reactor(s) in which the main reaction and/or the sulfhydrolysis reaction takes place may be fed with fresh H₂S and/or recycled H₂S. The recycled H₂S may be the unreacted H₂S recovered on conclusion of one or more of steps B), C), D) and/or F), preferably on conclusion of steps C) and/or F).

Furthermore, it has been observed that the preparation of mercaptan(s) from at least one alcohol and H₂S can lead to the formation of dialkyl disulfide impurities (noted DADS and of the type R—S—S—R).

Without wishing to be bound by theory, these DADS could be formed according to the following balanced equation (7) (example starting from methanol):

2CH₃SH+CH₃OH→CH₃—S—S—CH₃+CH₄+H₂O (7)

These DADS end up with the dialkyl sulfide(s) and then in the reactor in which the sulfhydrolysis is performed. Over time, they may lead to pressure losses on the catalyst and/or to clogging in this reactor or further downstream in the process. This phenomenon might be explained by coking of the catalyst due to parasitic or secondary reactions of the sulfhydrolysis reaction with the DADS. The sulfur products or impurities formed by such reactions may accumulate and create blockages in industrial facilities, giving rise to obvious safety and production problems. This may be all the more problematic when it comes to recycling the outlet stream from the sulfhydrolysis reactor into the main mercaptan production unit.

In particular, when methyl mercaptan is formed from methanol and H₂S, dimethyl disulfide (DMDS) may be formed secondarily. When this DMDS is present during the sulfhydrolysis reaction, it has been observed that the facilities (in the reactor and downstream of the sulfhydrolysis reactor) are clogged with sulfur impurities.

Surprisingly, when sulfhydrolysis is performed on a dialkyl sulfide previously separated from the DADS, these phenomena of pressure losses and/or clogging are no longer observed. Thus, in particular, said process comprises the following steps:
A) H₂S and at least one alcohol are introduced into a first reactor;
B) the H₂S and said at least one alcohol are reacted to obtain an outlet stream comprising at least one mercaptan, at least one dialkyl sulfide and at least one dialkyl disulfide (DADS) and possibly unreacted H₂S;
C) the outlet stream obtained from step B) is separated into:
   a stream F1 comprising the mercaptan(s),
   a stream F2 comprising the dialkyl sulfide(s), and the DADS, and
   optionally a stream F3 comprising H₂S;
D) a purification step is performed on stream F2 in order to separate:
   a stream F2′ comprising the dialkyl sulfide(s); and
   the DADS(s);
E) stream F2′ is introduced with H₂S into a second reactor; said reactor comprising a catalyst treated according to the treatment as defined above (i.e. the treatment according to step i) above);
F) a sulfhydrolysis reaction of the dialkyl sulfide(s) with H₂S is performed to obtain an outlet stream F4 comprising said mercaptan(s), and possibly unreacted H₂S;
G) optionally, stream F4 obtained from step F) is recycled into step A).

In step D), a step for purification of stream F2 is notably performed so as to obtain:
a stream F2′ comprising the dialkyl sulfide(s); and
the DADS(s) or a stream F5 comprising the DADS(s).

According to this embodiment, step D) is notably a step of purification by separation, on the one hand, of the dialkyl sulfide(s) and, on the other hand, of the DADS(s) and optionally of the heavy impurities present in stream F2. Step D) is more particularly a step of separating dimethyl sulfide from the DMDS present in stream F2.

Stream F2 may comprise at least 80%, preferably at least 95%, by weight of dialkyl sulfide(s) relative to the total weight of stream F2. For example, stream F2 comprises between 95% and 99.9% by weight of dialkyl sulfide(s) relative to the total weight of stream F2.

Stream F2 may comprise between 0.1% and 20%, preferably between 0.1% and 5%, by weight of DADS relative to the total weight of stream F2.

Said purification step may correspond to at least one distillation step, or to at least one step of adsorption of the DADS(s) on a porous support (for example on active charcoal), or to at least one step of selective extraction of the DADS(s) using a solvent that is immiscible with said dialkyl sulfide(s) and miscible with said DADS(s), for example water. These various techniques may be combined together.

Entirely preferably, said purification step corresponds to at least one distillation step, preferentially to a single distillation step. According to one embodiment, said purification step consists of a single distillation step.

The pressure during the distillation may be between 0.05 and 75 bar absolute, preferably between 1 and 30 bar absolute, more particularly between 5 and 15 bar absolute, for example approximately 10, 11, 12, 13, 14 or 15 bar absolute.

The distillation temperature may be between 20° C. and 250° C., preferably between 60° C. and 200° C., more preferentially between 100° C. and 180° C.

The column head temperature may be between 20° C. and 250° C., preferably between 60° C. and 200° C., more preferentially between 100° C. and 180° C. In particular, the column head is at a temperature of between 100° C. and 180° C. and at a pressure of between 5 and 15 bar absolute.

The column bottom temperature may be between 50° C. and 300° C., preferably between 100° C. and 250° C. In particular, the column bottom temperature is higher than the column head temperature.

Part of stream F2′ may be returned as reflux into the distillation column (stream F6 below). The mass reflux ratio (F6/F2′) in the column may be between 0 and 0.99, preferably between 0 and 0.70.

Preferably, stream F2′ is recovered at the top of the column and the DADS (or stream F5) is recovered at the bottom.

As indicated above, stream F3 may be combined with stream F2, in which case it may undergo the purification step D).

In the case of distillation, the H₂S ends up at the top with stream F2′ and may be sent with it to the sulfhydrolysis reactor.

The distillation may be performed in any known type of distillation column. It may be a column with trays (for example trays with caps, trays with valves or perforated trays) or with packing (for example with bulk or structured packing). The distillation may be performed in a tray column, preferably comprising between 5 and 50 trays, more preferentially between 10 and 40 trays, for example between 10 and 30 trays. The distillation may also be performed in a partition column (“DWC” or Divided Wall Column). The partition may be fixed or mobile, for example with structured or bulk packing.

On conclusion of step D), stream F2′ notably comprises less than 1000 ppm (by mass), preferably less than 500 ppm, more preferentially less than 100 ppm or even less than 10 ppm of DADS. In particular, stream F2′ comprises strictly less than 1000 ppm (by mass).

The mercaptan(s) may be recovered from stream F1 and/or stream F4, preferably by recovery from stream F1.

The mercaptan(s) may be recovered from stream F1 and/or stream F4, preferably by recovery from stream F1.

The separation step C) may be performed via conventional methods, preferably by distillation (notably under reduced pressure). During the distillation, the pressure may be between 1 and 40 bar absolute, and/or the temperature may be between 20° C. and 100° C. at the top of the column, and between 40° C. and 200° C. at the bottom of the column. For example, the distillation may take place at a pressure of between 0.1 bar and 10 bar absolute, notably between 1 and 10 bar absolute.

Prior to step C), the outlet stream from step B) may undergo one or more purification steps, for example to remove any water and/or H₂S that may be present. This/these purification step(s) may be performed by conventional decantation and/or distillation.

The introduction of stream F4 into the first reactor has no influence on the main reaction between at least one alcohol and H₂S. Furthermore, the H₂S which may be obtained from step F) may thus be totally recycled into step A). Such recycling notably has the advantage of having only one H₂S inlet for the entire mercaptan production process, for example at the inlet of the sulfhydrolysis reactor.

Thus, the sulfhydrolysis process according to the invention integrated into an industrial mercaptan production facility makes it possible to efficiently reprocess the dialkyl sulfide byproducts as products of interest and advantageously to recycle the H₂S and, if necessary, to prevent any clogging so as to operate safely and continuously.

The mercaptans produced will be the result of the main reaction and of the sulfhydrolysis reaction, which increases the productivity.

### DESCRIPTION OF THE FIGURES

FIG. 1 graphically represents an embodiment of the sulfhydrolysis catalyst treatment according to the invention. Steps 1) and 2) are represented as a function of temperature and time.

FIG. 2 schematically shows a methyl mercaptan production unit incorporating the sulfhydrolysis process according to the invention.

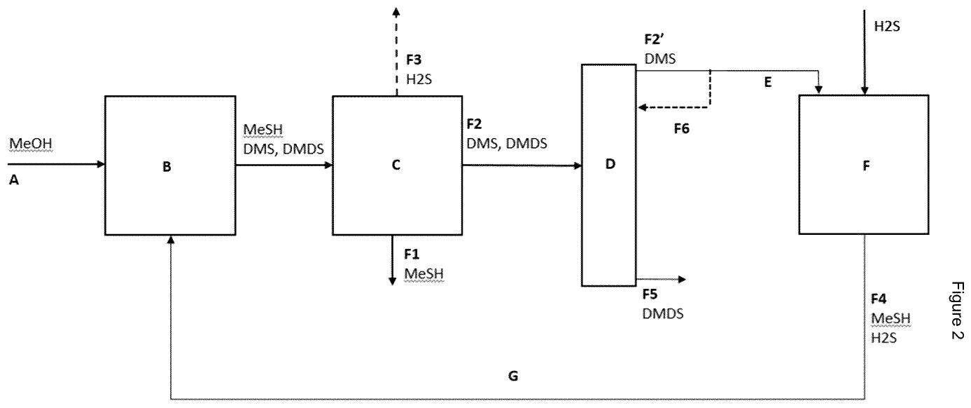

In step A), H₂S and methanol are placed in the reactor in which step B) takes place, to form a stream comprising methyl mercaptan and dimethyl sulfide (DMS). This stream undergoes separation in step C) to obtain:
a stream F1 comprising methyl mercaptan,
a stream F2 comprising dimethyl sulfide, and
   an optional stream F3 comprising H₂S may be obtained.

Stream F2 is distilled to separate DMS from its DMDS impurity and to obtain a stream F2′ at the top of the column comprising purified DMS. A stream F5 comprising the DMDS is obtained at the bottom of the column. A stream F6, part of F2′, is returned to the distillation column. Stream F2′ is introduced with a stream of H₂S, into a reactor comprising a catalyst treated according to the invention to perform the sulfhydrolysis reaction (step F)). An outlet stream F4 comprising methyl mercaptan and H₂S is obtained. Stream F4 is fully recycled into step A). The examples below are given for illustrative purposes and do not limit the present invention.

### EXAMPLES

### Example 1: Sulfhydrolysis Process Using an Activated Catalyst According to the Invention

### 1—Catalyst Treatment:

All the steps are performed at P=1 bar abs.

30 mL of catalyst are fed into a 316Tl stainless steel reactor. The catalyst is in extruded ⅛ form and its trade name is TCC101 from Axens, internal radius 7.7 mm.

It is a Y-type zeolite, with a lattice parameter of between 24.30 and 24.70 Å, an Si/Al ratio of between 2.5 and 15 and comprising less than 10% by weight of Na₂O, relative to the total weight of the zeolite.

The treatment begins with a step of heating under N₂, with a temperature rise of 5° C./min at 20 NL/h, and the catalyst is then maintained for 1 h at 120° C.

The dinitrogen is then shut off.

Pure H₂S is injected at 20 NL/h with a temperature rise to 325° C. at 5° C./min, and the catalyst is then maintained for 1 h at 325° C. under H₂S.

The treatment is then complete.

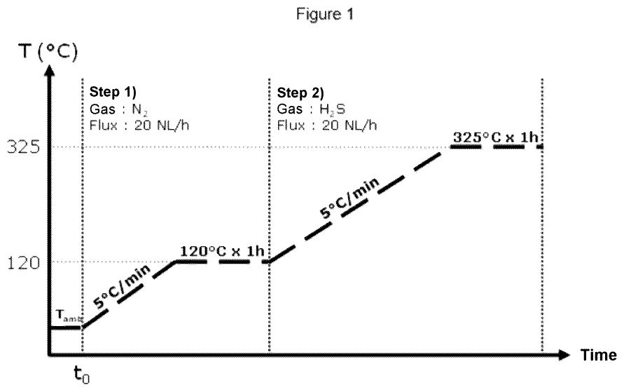

This treatment protocol is illustrated in FIG. 1.

### 2—Sulfhydrolysis Reaction:

The sulfhydrolysis reaction is then performed in the reactor in the presence of the catalyst as activated above or not.

The stream of pure H₂S is maintained and the reaction temperature is 325° C.

The reaction mixture (H₂S/DMS=4/1 molar) is injected at 325° C. with a pressure rise to 9 bar abs, with a residence time of 40.5 seconds.

The results obtained are given in Table 1 below:

TABLE 1

| | Without catalyst | With catalyst |
|---|---|---|
| T = 325° C. | treatment | treatment |
| DMS conversion (%) | 37 | 47 |
| MeSH selectivity (%) | 99.8 | 99.8 |

A 10% increase in DMS conversion is observed, with no loss of selectivity.

### Example 2: Separation of DMDS Impurity Prior to the Sulfhydrolysis Reaction

### Test A:

A dimethyl sulfide (DMS) sulfhydrolysis reaction is performed in the presence or absence of DMDS, as follows.

DMS comprising the following (relative to the total weight DMS+DMDS) is introduced into a reactor:
either 0.02% by weight of DMDS;
or 14% by weight of DMDS.

The sulfhydrolysis reaction is performed under the following conditions.

The catalyst used is TCC101® from Axens (catalyst in ⅛ extruded form with an internal radius of 7.7 mm).

It is a Y-type zeolite with a lattice parameter of between 24.30 and 24.70 Å, an Si/Al ratio of between 2.5 and 15 and comprising less than 10% by weight of Na₂O.

The reaction temperature is 340° C. and the pressure is 25 barg.

The H₂S/DMS mole ratio is 30.0.

Result: With DMS comprising 14% by weight of DMDS, clogging was observed in the reactor after a few hours, whereas with DMS comprising 0.02% by weight of DMDS, no clogging was observed after 1000 hours.

This test demonstrates the role of the DMDS impurity in the clogging phenomena.

### Test B:

Before performing the sulfhydrolysis reaction as described in test A, the introduced DMS is first separated or not from the DMDS impurity by distillation.

The distillation conditions are as follows:

A column with a number of trays of between 10 and 20 is used.

The column head pressure is between 5 and 15 barg.

The column head temperature is between 130° C. and 140° C.

The column bottom temperature is between 135° C. and 150° C.

The reflux flow rate is between 900 kg/h and 1200 kg/h.

### Composition of the Entering Stream:

TABLE 2

| Mass composition of | | |
|---|---|---|
| the stream introduced | Without | With |
| into the sulfhydrolysis | distillation | Distillation |
| reactor | (F2) | (F2′) |
| % DMS | 98.8% | 99.3% |
| % MeSH | 0.07% | 0.08% |
| % H₂O | 0.60% | 0.60% |
| % DMDS | 0.53% | 0.02% |

Without prior distillation, clogging occurs after 100 h of running the sulfhydrolysis. With prior distillation, no clogging is observed after 1000 h.

1. A process for preparing at least one mercaptan, comprising the following steps:
i) treatment of a catalyst for the sulfhydrolysis of at least one dialkyl sulfide, comprising the following steps:
1) heating said catalyst, preferably in the presence of an inert gas; and
2) placing said catalyst in contact with hydrogen sulfide (H₂S); and then
ii) a sulfhydrolysis reaction in which at least one dialkyl sulfide is reacted with H₂S in the presence of said catalyst treated according to step i), to obtain at least one mercaptan;
said catalyst being a zeolite.

2. The process according to claim 1, in which said catalyst is a Y-type zeolite.

3. The process according to claim 1, in which said catalyst comprises between 0% and 10% by weight of promoter, preferably between 0% and 2% by weight of promoter, for example between 0.01% and 2% by weight of promoter, relative to the total weight of said catalyst.

4. The preparation process according to claim 1, in which the treatment of the catalyst according to step i) is performed in the reactor in which the sulfhydrolysis of the dialkyl sulfide according to step ii) is then performed.

5. The preparation process according to claim 1, in which the heating step 1) is performed in the presence of an inert gas and at a temperature of between 70° C. and 350° C., more particularly between 80° C. and 250° C., and preferably between 80° C. and 150° C. and at a pressure of between 0.1 and 50 bar absolute, in particular between 0.1 and 10 bar absolute, preferably between 0.8 and 2 bar absolute.

6. The preparation process according to claim 1, in which step 2) of placing in contact with H₂S is performed at a temperature of between 20° C. and 450° C., for example between 250° C. and 400° C., preferably between 320° C. and 370° C., and at a pressure of between 0.1 and 50 bar absolute, more particularly between 1 and 20 bar absolute, for example between 1 and 15 bar absolute.

7. The preparation process according to claim 1, in which, in the sulfhydrolysis reaction, the H₂S/dialkyl sulfide mole ratio is between 0.1/1 and 50/1, preferably between 2/1 and 20/1, more preferentially between 2/1 and 8/1.

8. The preparation process according to claim 1, in which the dialkyl sulfide is chosen from the group consisting of: dimethyl sulfide, diethyl sulfide, dioctyl sulfide, didodecyl sulfide and methyl ethyl sulfide, preferably dimethyl sulfide.

9. A process for preparing at least one mercaptan, preferably continuously, comprising the following steps:
A) H₂S and at least one alcohol are introduced into a first reactor;
B) the H₂S and said at least one alcohol are reacted to obtain an outlet stream comprising at least one mercaptan and at least one dialkyl sulfide and possibly H₂S;
C) the stream comprising at least one mercaptan, at least one dialkyl sulfide and possibly H₂S is separated into:
a stream F1 comprising the mercaptan(s),
a stream F2 comprising the dialkyl sulfide(s), and
optionally a stream F3 comprising H₂S;
D) optionally, stream F2 is purified to obtain a stream F2′ enriched in dialkyl sulfide(s);
E) stream F2 or F2′ is introduced with H₂S into a second reactor, said reactor comprising a catalyst treated according to the treatment of step i) as claimed in claim 1;
F) a sulfhydrolysis reaction of the dialkyl sulfide(s) with H₂S is performed to obtain an outlet stream F4 comprising said mercaptan(s) and possibly H₂S;
G) optionally, stream F4 obtained from step F) is recycled into step A).
